(19) United States
(12) Patent Application Publication
(10) Pub. No.: US 2002/0010458 A1
(43) Pub. Date: Jan. 24, 2002

(54) ELECTRICAL SURGICAL APPARATUS INCLUDING PERSONAL COMPUTER

(76) Inventors: ALEX URICH, MISSION VIEJO, CA (US); MICHAEL CURTIS, LAKE FOREST, CA (US); GREG PENA, WEST COVINA, CA (US); BRENT MARTINEZ, WHITTIER, CA (US)

Correspondence Address: KLIMA AND PEZZLO, P.O. BOX 2855, STAFFORD, VA, 22554, US

(21) Appl. No.: 09/179,863

(22) Filed: Oct. 28, 1998

(51) Int. Cl. A61B 17/00; A61B 17/32

(52) U.S. Cl. 606/1; 606/169

(57) ABSTRACT

A phacoemulsification apparatus including a personal computer for displaying, storing and transmitting data relating to a surgical operation. Preferably, the personal computer is operated by MICROSOFT WINDOWS 95 or 98 software to take advantage of various communication applications.

### FIELD OF THE INVENTION

An electrical surgical apparatus including a personal computer for displaying, recording, and transmitting surgical data parameters and audio/video information regarding a particular surgery, and methods of operating the electrical surgical apparatus and methods of surgery. A preferred embodiment utilizes a personal computer operating on WINDOWS 95 and WINDOWS 98 software. An example of an electrical surgical apparatus according to the present invention is a phacoemulsification apparatus for conducting eye surgery.

### BACKGROUND OF THE INVENTION

There exists many surgical instruments and apparatus for conducting surgical procedures on animals and humans. Many of these instruments are electrical surgical instruments and include various electrical devices for powering and controlling the surgical instruments.

Currently, most electrical surgical instruments are custom configured and designed for one or more particular surgical applications. These surgical instruments utilize analog and/or digital sensing and control equipment and devices. The outputs from these sensors and controls can be visually displayed on a monitor associated with the electrical surgical instrument.

The information displayed is based on custom software developed with the operating system of the electrical surgical instrument. An instrument designer and/or programmer develops the particular custom hardware and software configurations for displaying the desired information.

In the past, it is believed that electrical surgical apparatus have not been interfaced with a personal computer being operated by WINDOWS 95 and WINDOWS 98 software. The present invention is designed and configured to make the personal computer interface highly reliable and dependable, and suitable for use in controlling an electrical surgical apparatus.

### SUMMARY OF THE INVENTION

An object of the present invention is to provide an improved electrical surgical apparatus.

A second object of the present invention is to provide an electrical surgical apparatus including a personal computer.

A third object of the present invention is to provide an electrical surgical apparatus including a personal computer operated by WINDOWS 95 and/or WINDOWS 98 software.

A fourth object of the present invention is to provide an electrical surgical apparatus including a personal computer for displaying and recording information relating to the surgery.

A fifth object of the present invention is to provide an electrical surgical apparatus including a personal computer and a highly reliable and dependable personal computer interface.

A sixth object of the present invention is to provide an electrical surgical apparatus including a personal computer configured for transmitting information relating to the surgery via telephone or internet systems.

A seventh object of the present invention is to provide an electrical surgical apparatus including an electrical surgical device acting as a master and a personal computer acting as a slave.

An eighth object of the present invention is to provide an improved phacoemulsification apparatus.

A ninth object of the present invention is to provide a phacoemulsification apparatus including a personal computer.

A tenth object of the present invention is to provide a phacoemulsification apparatus including a personal computer operated by WINDOWS 95 and/or WINDOWS 98 software.

The present invention relates to electrical surgical apparatus. The electrical surgical apparatus utilizes electrical energy or power for operating one or more functions of the apparatus. Typically, the apparatus would be connected via a power cord to a conventional power source (e.g. 110-115 volt, 15 amp ac wall outlet), or a non-conventional power source with additional power converting equipment.

The present invention particularly relates to electrical surgical apparatus configured to include 1) a control console; 2) separate surgical hand piece electrically connected (e.g. via cable or wire) to the control console; and 3) personal computer.

The control console will typically include a power source (e.g. power cord), power supply for powering the control console circuitry and hand piece, control circuit board(s), driver circuit board(s) for powering the hand piece, and other electrical equipment and devices for controlling and operating the separate surgical hand piece. The control console can be a separate stand alone unit, or can be integrated with the personal computer into a signal cabinet or case. A preferred configuration is with a movable stand supporting the console, and a movable arm supporting the personal computer with a display above the control console.

The surgical hand piece can include an electrical motor, transducer, ultrasonic transducer, magnetostrictive transducer, cooler, heater, electromagnetic generator, cauterizer, cutter, grinder, milling device, drill, laser, light source, and/or other suitable electrical components to conduct various surgical operations. A specific example is a hand piece for a phacoemulsification apparatus for conducting eye surgery for removal of the natural lens of an eye. The hand piece can use a needle driven by an ultrasonic or magnetostrictive transducer, or alternatively, a needle or probe provided with a high speed cutting bit for breaking the natural lens into tiny pieces to be removed through a small incision (e.g. by fluid irrigation and vacuum removal).

The personal computer utilized in the present invention is preferably an IBM compatible DOS based personal computer, WINDOWS 95 and/or WINDOWS 98 based personal computer, or MACINTOSH personal computer, since the current software is user friendly, and there exists substantial software applications for these computer systems. Most preferably, the personal computer is loaded and operated with Microsoft WINDOWS 95 and WINDOWS 98 software.

The personal computer is electrically connected to the console via an electrical cable connected to an isolated serial port of the personal computer. The electrical surgical apparatus is configured to send surgical parameters data from the console to the personal computer via the electrical cable connect to the isolated serial port of the personal computer. Further, the electrical surgical apparatus is configured so that the console acts as a master and the personal computer acts as a slave. If the personal computer experiences a crash, the operation of the control console is not affected.

In the application of the present invention to a phacoemulsification apparatus, the surgical parameters data communicated from the console to the personal computer, for example, may include ultrasonic power level of hand piece, pressure and/or flow within the infusion line, vacuum and/or flow within the aspiration line, flow within the liquid bypass line, peristaltic pump speed, phaco sleeve temperature, diathermy power, footswitch position, mode of operation, mode(s) of information display, modes of data storage and transmission, etc.

The personal computer interface provides the following functions, including:

1) collecting surgical parameters data in real time during the surgical procedure;

2) displaying a menu similar to the menu displayed by the control console such that different functions can be selected either by using a personal computer mouse, keyboard, touch screen, or the control console;

3) displaying the surgical parameters data as continuous graphs as a function of time;

4) storing the surgical parameters data in a data base file;

5) capturing a video signal from a camera placed on the surgical microscope and displaying the picture of the eye undergoing the surgical procedure; and

6) storing the picture on a recordable compact disc (CD) as a function of time.

When visualizing a surgical procedure, the personal computer will associate the name of the file from the compact disc with the name of the file in the data base. Both files are then synchronized in time and displayed in separate windows. Other pertinent data can be computed from the surgical parameters and displayed such as the temperature of the ultrasonic tip which is a function of the ultrasonic power, aspirated flow and incision leak.

### BRIEF DESCRIPTION OF THE DRAWINGS

FIG. 1 is a perspective view of a phacoemulsification apparatus according to the present invention.

FIG. 2 is a detailed front elevational view of the control console component of the phacoemulsification apparatus shown in FIG. 1.

FIG. 3 is a side elevation view of the control console shown in FIG. 2.

FIG. 4 is a rear elevational view of the control console shown in FIG. 2.

FIG. 2 is a top elevational view inside the control console shown in FIG. 2.

FIG. 6 is a perspective view of a movable pivot arm to be connected to the back of the stand to support the personal computer above the control console.

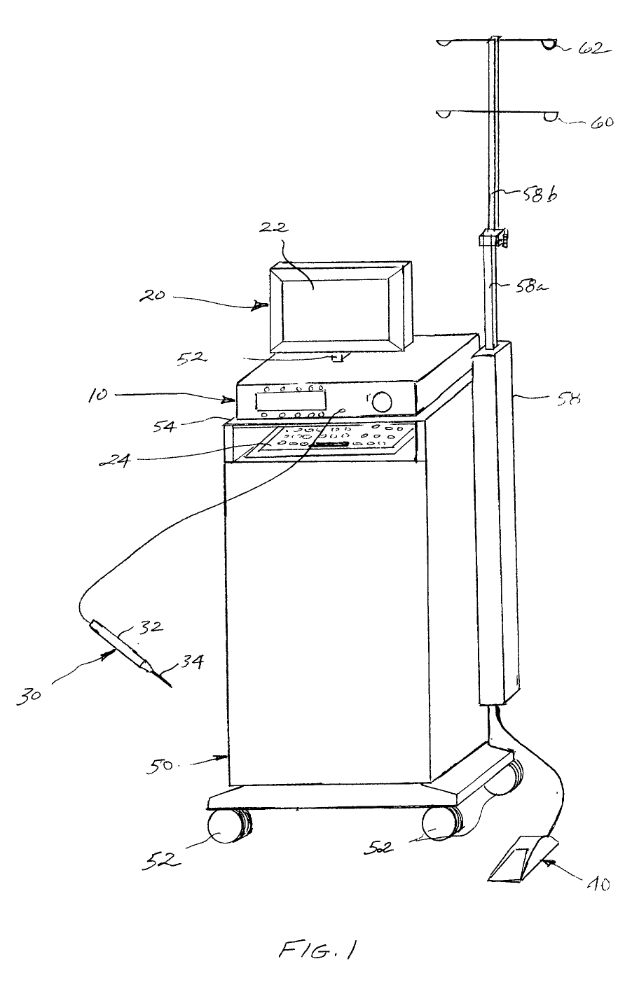

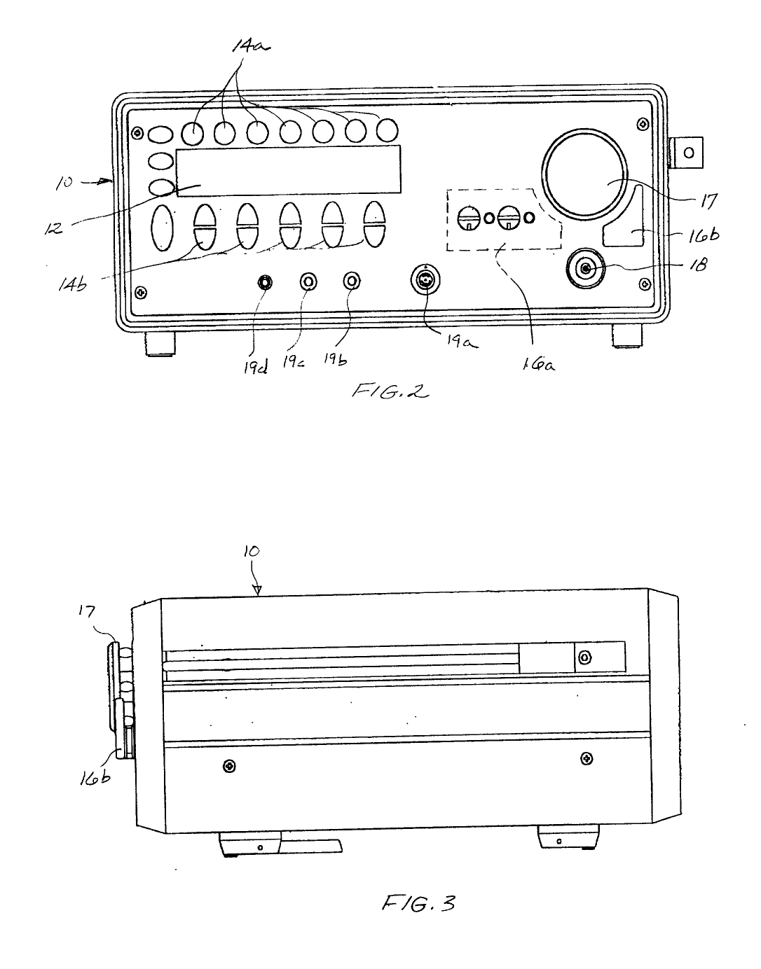

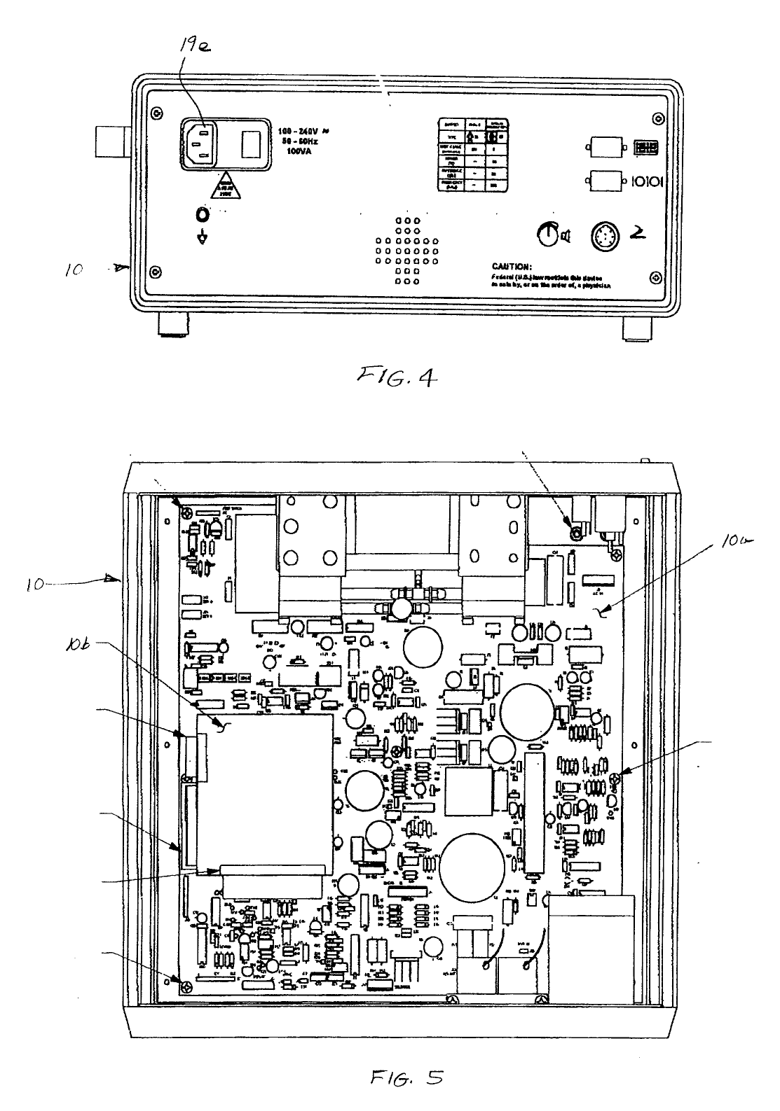

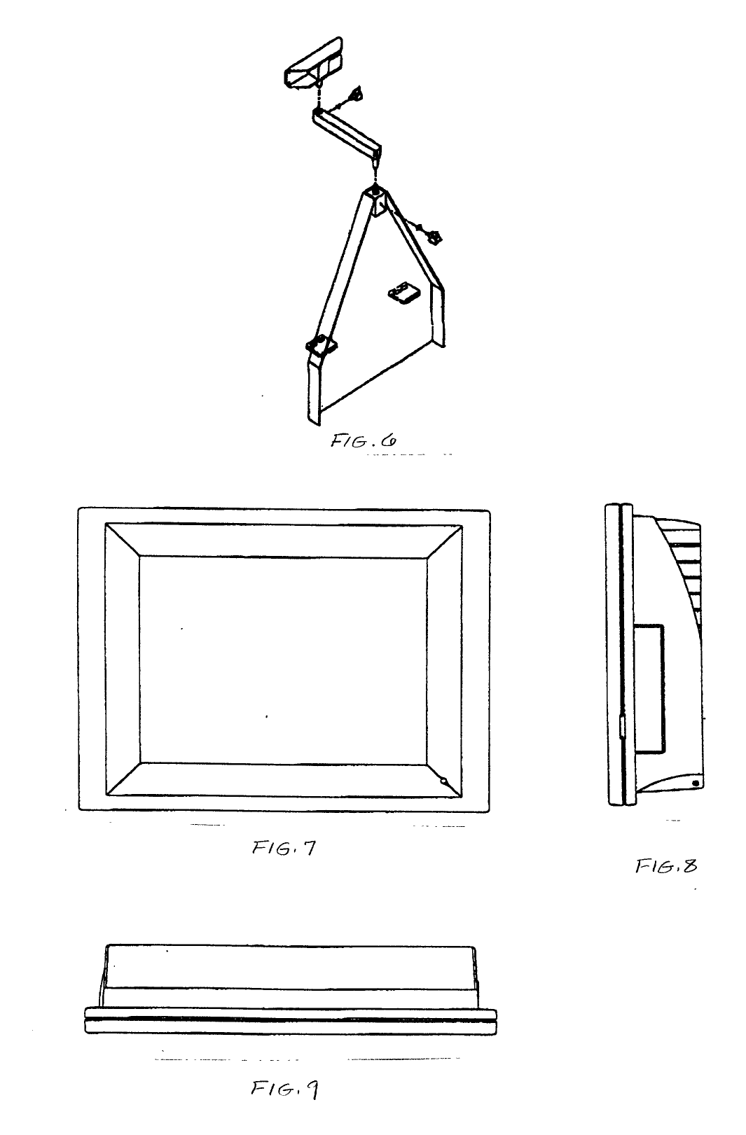

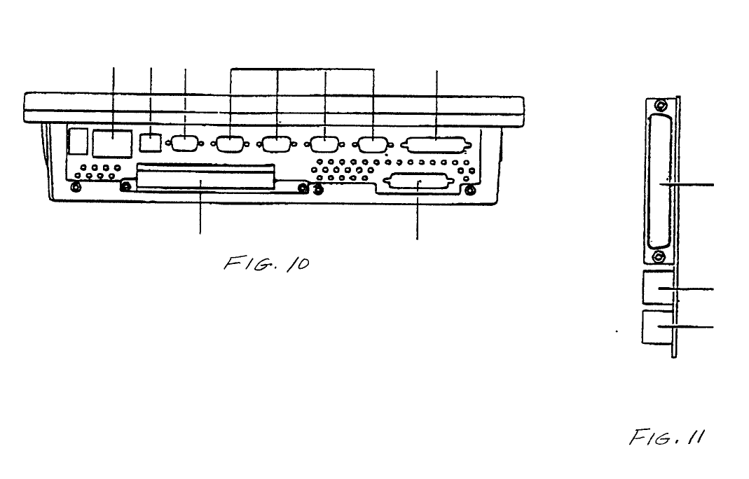

FIG. 7 is a front elevational view of the personal computer component of the phacoemulsification apparatus shown in FIG. 1.

FIG. 8 is a right side elevational view of the personal computer shown in FIG. 7.

FIG. 9 is a top planar view of the personal computer shown in FIG. 7.

FIG. 10 is a schematic bottom view showing the external connectors and ports of the personal computer shown in FIG. 7.

FIG. 11 is a schematic right side view showing the external connectors and ports of the personal computer shown in FIG. 7. in the phacoemulsification apparatus shown in FIG. 1.

### PREFERRED EMBODIMENTS

A particular example of an electrical surgical apparatus according to the present invention is a phacoemulsification apparatus to be described in detail below. The phacoemulsification apparatus comprises five (5) main components, including control console 10, personal computer 20, hand piece 30, foot pedal 40, and movable stand 50.

I. Control Console

The control console 10 is provided with a display 12, touch control buttons 14a and 14b, removable tubing cassette 16a and stationary tubing holder 16b, peristaltic pump 17, and vacuum sensor 18 provided on a front panel thereof, as shown in FIG. 2. The front panel is also provided with an ultrasonic supply jack 19a for connecting a cable leading to the hand piece 30, and additional ports 19b, 19c and 19d. The back panel of the control console 10 is provided with a conventional AC power jack

The console encloses various electrical components, including circuit board 10a and micro-controller 10b.

### II. Personal Computer

The personal computer 20 is provided with an integral screen 22 and remote keyboard 24 wired to the personal computer 20. The personal computer 20 is supported above the console 10 by a movable swing arm 42, as shown in FIG. 6, which is mounted to the back of the stand 40. The movable swing arm 42 allows a user to pivot the personal computer along a substantially vertical axis, and the movable swing arm can also be configured to provide additional axis of movement (e.g. tilting up and down, twisting, etc.).

The personal computer for use in the present invention can have various configurations. A notebook type personal computer is preferable due to its compact size. However, a personal computer 20 having the personal computer and screen integrated into a single unit with a remote keyboard 24 is currently most preferred.

An example of a suitable personal computer for use with a phacoemulsification apparatus according to the present invention is as follows:

1) IBM AT compatible personal computer with integrated super I/O;

2) VGA card;

3) touchscreen control;

4) ethernet 10BT with an external floppy interface;

5) built in 12.1 TFT Color LCD with a MAX of 800×600 resolution;

6) Pentium MMX 233 megahertz processor with 128K cache, 32 megabytes of onboard memory;

7) 2 megabytes video memory;

8) 6 gigabytes hard disk drive;

9) one (1) PCI slot;

10) SNAZZI video capture card for the one (1) PCI slot;

11) optional external CD Rom record

The SNAZZI video capture card is independent of the built in VGA card and provides hardware compression at 11 MB/minute. This equates roughly to 60 minutes of video on one compact disc. The quality of this capture and compression is excellent.

The system will, as an option, also support an external CD Rom recorder. This will allow the digital storage of videos captured using the SNAZZI card. The Hard Drive will be capable of storing roughly six (6) hours of video (363 minutes).

The personal computer 20 is provided with the following I/O arrangement, as shown in FIGS. 5 and 6.

20a—AC inlet

20b—Ethernet phone jack

20c—Expansion outlet

20d—VGA port IF

20e—4×serial ports

20f4×digital inputs/outputs

20g—Parallel port

20h—Keyboard

20i—PS/2 mouse

20j—External FDD

The personal computer is preferably programmed to communicate with the control console by using a check sum approach. Specifically, the amount of bits of data being sent precedes the actual data being sent. This ensures that the data being sent from the personal computer is correct as opposed to having errors from the personal computer crashing. This packaging of information provides redundancy to allow highly reliable communication between the personal computer and control console.

The control console can also provide with voice activation hardware and/or software to allow a user to voice active certain operations (e.g. operating mode) of the control console.

III. Phacoemulsification Hand Piece

The phacoemulsification hand piece 30 can be a conventional ultrasonic type hand piece including an ultrasonic transducer located within a housing 32. An ultrasonically driven needle is connected to the hand piece 30 and provided with an outer resilient sleeve (e.g. made of highly resilient silicone material).

IV. Foot Pedal

A conventional multi-position foot pedal can be used with the phacoemulsification apparatus according to the present invention.

V. Stand

The stand 50 is custom designed to support the other components of the phacoemulsification apparatus to facilitate easy and effective operation thereof.

The stand 50 is provided with a set of coaster wheels 52 (e.g. locking and non-locking type combinations) to allow a user to maneuver the phacoemulsification into position during an operation or later for storage.

The stand 50 is provided with an upper shelf 54 defining a top panel of the stand 50 for supporting the console 10 thereon. A lower inner shelf 56 is provided for supporting the key board 24.

The stand 50 is provided with a side support 58 including fixed telescoping portion 58a, and adjustable height telescoping portion 58b. The telescoping portion 58b is provided with a pair of irrigation bag loop supports 60 and 62 each having a pair of supporting loops. The loop supports 60 and 62 are located at different heights to provided the potential implementation of multiple irrigation bags in some system configurations (e.g. to provide variable or adjustable irrigation pressure).

We claim:

1. A phacoemulsification apparatus, comprising:
a control console;
a personal computer interfaced to said control console, said personal computer including a visual display and a keyboard, said personal computer loaded and operating by MICROSOFT WINDOWS 95 or WINDOWS 98 software.

2. An apparatus according to claim 1, wherein said personal computer is directly connected by hard wiring to said electrical surgical device.

3. An apparatus according to claim 1, wherein said personal computer is remotely connected by hard wiring to said electrical surgical device.

4. An apparatus according to claim 1, wherein said personal computer is remotely connected by a non-wire communication link to said electrical surgical device.

5. An apparatus according to claim 4, wherein said non-wire communication link includes a satellite link.

6. An apparatus according to claim 5, wherein said non-wire communicating link includes electromagnetic wave communication.

7. An apparatus according to claim 1, wherein said personal computer is connected to said control console by an electrical cable connected to an isolated serial port of said personal computer.

8. An apparatus according to claim 1, wherein said personal computer is programmed to communicate with said control console by sending packets of data with check sums.
